(19) United States
(12) Patent Application Publication
(10) Pub. No.: US 2016/0106681 A1
(43) Pub. Date: Apr. 21, 2016

(54) CONTROLLED RELEASE OF VEGETARIAN PORE-SEALING CAPSULE IN STOMACH, DUODENUM, JEJUNUM, ILEUM AND COLON

(71) Applicant: bio-Peptide Enhancer Tech Inc., Ltd., New Taipei City (TW)

(72) Inventor: Chao-Hsiang Chang, New Taipei City (TW)

(21) Appl. No.: 14/882,634

(22) Filed: Oct. 14, 2015

(30) Foreign Application Priority Data
Oct. 15, 2014 (TW) 103135670

(51) Int. Cl. A61K 9/48; A61K 9/00

(52) U.S. Cl. CPC A61K 9/4808; A61K 9/4858; A61K 9/4866; A61K 9/485; A61K 9/0053

(57) ABSTRACT

A vegetarian pore-sealing capsule is provided. The pore-sealing capsule may release active substances in a gastrointestinal tract, including stomach, duodenum, jejunum, ileum and colon, under control. The pore-sealing capsule includes a capsule shell having one or more pores and a pore-sealing material for sealing at least one of the pores. When the pore-sealing capsule is located at a destination inside the gastrointestinal tract, the pore-sealing material will leave the pores.

### CROSS-REFERENCE TO RELATED APPLICATION

This application claims the priority benefit of Taiwan application serial no. 103135670, filed Oct. 15, 2014, the full disclosure of which is incorporated herein by reference.

### BACKGROUND

1. Technical Field

The disclosure relates to a capsule. More particularly, the disclosure relates to a pore-sealing capsule.

2. Description of Related Art

There are many kinds of controlled-release formulations designed for releasing active substances in the gastrointestinal tract under control. These controlled-release formulations are quite diversified and complex. The discharge time of these controlled-release formulations from the gastrointestinal tract are affected by physiological rhythm, life style, aging, and disease. Therefore, when a formulation, such as a tablet, having a specific use is developed, many designs, changes, and tests are needed. For example, designs of the formulation, changes of prescription, adjustments of physical and chemical properties for mass-production, dissolution tests, deviation tests, and stability tests are all needed for developing a new formulation. Even for the same simple or compound prescription of active substances, when the doses are increase by 1.2-4 times, the designs and test results for previous doses are also hard to be used as a reference, and thus the designs and the tests have to be redone. Accordingly, long development time and high labor costs are wasted, and the willingness of pharmaceutical and biotechnology companies is thus reduced to develop differentiated and novel products.

Moreover, since the choices of various doses of drugs are few, it is hard for clinicians and pharmacists to choose suitable doses for individual patients according to their weights or body surface area, and the clinical effects and the side effects are hard to be evaluated and controlled. Especially for the formulations using compound prescription, the difficult development and the high cost limit the small pharmaceutical and biotechnology companies to develop differentiated and characteristic products.

### SUMMARY

In one aspect, a vegetarian pore-sealing capsule released in a gastrointestinal tract under control is provided. The vegetarian pore-sealing capsule comprises a capsule shell having one or more pores and a pore-sealing material located in at least one of the pores for sealing the pore. The pore-sealing material will leave the pore when the pore-sealing capsule is located in a destination of the gastrointestinal tract.

According to an embodiment, the pore-sealing material may be decomposed by flora in the gastrointestinal tract.

According to another embodiment, the pore-sealing material may be decomposed by a digestive enzyme in the gastrointestinal tract. The pore-sealing material may be a carbohydrate, a protein, or a lipid, for example.

According to another embodiment, the pore-sealing material may be disintegrated at a certain pH value in the gastrointestinal tract.

According to another embodiment, the pore-sealing material is melted at a certain temperature in the gastrointestinal tract. The pore-sealing material may be a fatty acid, for example. Optionally, the pore-sealing material further comprises a surfactant selected from a group consisting of sodium dodecyl sulfate, sodium laurate, vitamin E, a bile acid, a bile salt, lecithin, and a combination thereof.

According to another embodiment, the pore-sealing capsule may further comprise carriers located in the capsule shell.

According to another embodiment, at least one of the carriers comprises an expander.

According to an embodiment, the expander is hydroxypropyl cellulose (HPC), sodium starch glycolate (SSG), polyvinylpyrrolidone (PVPP), hydroxypropyl starch, cross-linked sodium carboxymethyl cellulose, or a combination thereof.

According to another embodiment, the expander is a base and an acid isolated from each other. The base may be NaHCO₃, KHCO₃, or a combination thereof. The acid may be citric acid, malic acid, tartaric acid, phosphoric acid, lactic acid, gluconic acid, glucuronic acid, vitamin C, acetic acid, salicylic acid, or a combination thereof.

According to an embodiment, at least one of the carriers comprises an absorbefacient. The absorbefacient may be a surfactant, a fatty acid, an alcohol, an azone, chitosan, a phospholipid, a rubefacient, and piperine.

According to an embodiment, at least one of the carriers comprises a mineral. The mineral may be ferromagnetic, paramagnetic or diamagnetic.

Accordingly, depending on the needs and the desired destination in the gastrointestinal tract, a proper pore-sealing material may be chosen to release a drug in the pore-sealing capsule under control.

In addition, the material of the vegetarian pore-sealing capsule is a vegetarian material, such as a material from plant or a non-animal source.

The foregoing presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIG. 1 is a diagram of a pore-sealing capsule according to an embodiment of this invention.

FIG. 2 is a diagram of a pore-sealing capsule according to another embodiment of this invention.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

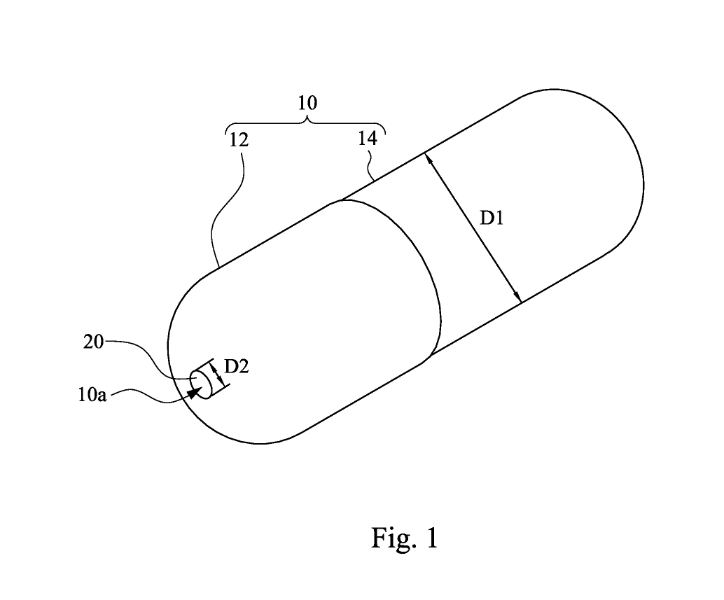

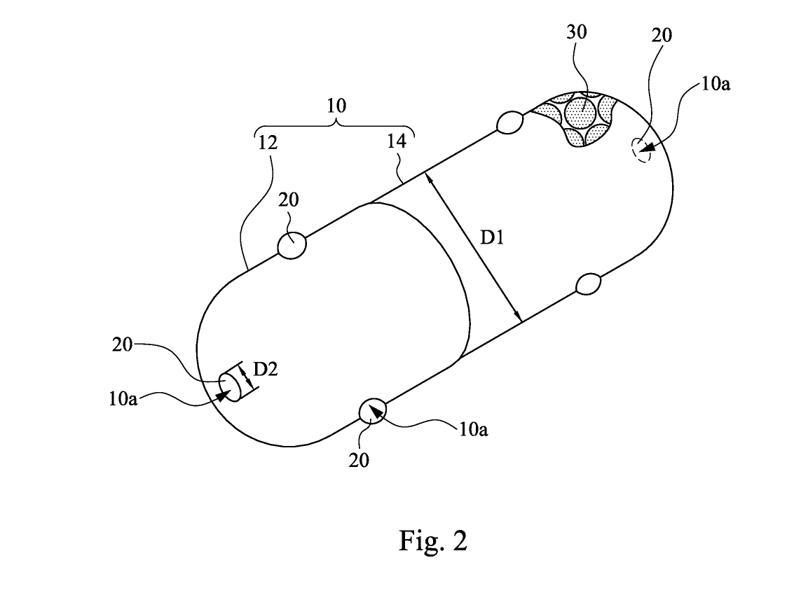

FIG. 1 is a diagram of a pore-sealing capsule according to an embodiment of this invention. In FIG. 1, a pore-sealing capsule comprises a capsule shell 10 having at least a pore 10a thereon, and a pore-sealing material 20 filled in the pore 10a. At least an active substance may be encapsulated in the pore-sealing capsule in a form of powder or carried by carriers, such as pellets, as shown in FIG. 2.

FIG. 2 is a diagram of a pore-sealing capsule according to another embodiment of this invention. In FIG. 2, the pore-sealing capsule may further comprises carriers 30 encapsulated in the pore-sealing capsule. The carriers 30 may be used to carry active substances, excipients, and other additives.

In FIGS. 1 and 2, the capsule shell 10 comprises a head part 12 and a body part 14. The head part 12 and the body part 14 are intermeshed to form a close space. The capsule shell 10 is made of a material having good ductility, malleability, compression resistance and hygroscopicity, as well as has a suitable thickness to avoid rupture due to fast softening in the gastrointestinal tract.

In some embodiments, the material of the capsule shell 10 comprises a plant-derived material, hydroxypropylmethyl cellulose, or any combinations thereof, but is not limited thereto. The plant-derived material may be corn starch, tapioca starch, yam starch, potato starch, or polysaccharides, for example. The polysaccharides may be extracted from plants, or obtained by fermentation of yeasts or fungi. The polysaccharides may be a cellulose (nature or modified), a glucan, or a mucopolysaccharides.

In some embodiments, the thickness of the capsule shell 10 is 0.11-0.115 mm. The diameter D1 of the capsule shell 10 is better to be at most 6 mm, such as number 3, 4, or 5 capsules in international standard. Therefore, the pore-sealing capsule may smoothly pass through pylorus of stomach under a physiological state of not eating into the intestinal tract.

The capsule shell 10 has one or more pores 10a penetrating through the capsule shell 10, so that the active substances in the capsule shell 10 may be released from the inside of the capsule shell 10 to the outside of the capsule shell 10. The pores 10a may be formed by drilling. The drilling method may be using a needle or a punch mold to perforate the capsule shell 10, but is not limited thereto. The shape of the pore 10a may be circle, oval, or other suitable shapes, and is not limited thereto.

The diameter D2 of the pore 10a may be determined according to the actual needs. In some embodiments, the diameter D2 of the pore 10a is at most 0.055 mm to avoid the leakage of powder from the capsule shell 10 through the pore 10a, since the size of the powder is usually larger than 0.055 mm, i.e. the sieve of number 270 in US standard. In some other embodiments, the diameter D2 of the pore 10a is at most 0.15 mm to avoid the leakage of pellets from the capsule shell 10 through the pore 10a, since the size of the pellets is usually larger than 0.15 mm, i.e. the sieve of number 100 in US standard.

The number of pore 10a also may be determined according to the actual needs, as long as the capsule shell 10 will not be broken before reaching the destination of the gastrointestinal tract. In some embodiments, the number of pore 10a is 1-6. As shown in FIG. 1, the number of the pore 10a is one. In FIG. 2, the number of the pores 10a is six. Two of the pores 10a are respectively located on the terminals of the head part 12 and the body part 14, and the rest four pores 10a are respectively located on the side walls of the head part 12 and the body part 14. Of course, the positions of pores 10a may be arbitrarily changed, and are not limited by the examples above. Moreover, not all of the pores 10a have to be filled with the pore-sealing material 20. It may be that only some of the pores 10a are filled with the pore-sealing material 20.

The pore-sealing material 20 is filled in at least one of the pores 10a. The pore-sealing material 20 may be protruding from, recessed below, or level with the outer surface of the capsule shell 10. In FIG. 2, the pore-sealing material 20 is protruding from the surface of the capsule shell 10 and in a dome shape, for example. Especially, the pore-sealing material 20 will leave the pores 10a when the pore-sealing capsule is located at a certain position in the gastrointestinal tract to release the active substances inside the pore-sealing capsule under control.

There are many ways can let the pore-sealing material 20 leave the pores 10a, and some examples are described below. In some embodiments, the inner pressure of the pore-sealing capsule may be increased by an expander inside the pore-sealing capsule to push the pore-sealing material 20 outward through the pores 10a, and the pore-sealing material 20 thus can leave the pores 10a to decrease the pressure inside the pore-sealing capsule. The increasing rate of the inner pressure of the pore-sealing capsule is better to be carefully controlled to avoid the pore-sealing capsule from breaking due to the rapidly increased pressure in the pore-sealing capsule. Therefore, the content of the expander may be 1-9 wt % of the carriers 30. That is, only a portion of the carriers 30 are used to carry the expander.

The expander may be either of dissolved expansion type or gas generating type. The expander of dissolved expansion type may be hydroxypropyl cellulose (HPC), sodium starch glycolate (SSG), polyvinylpyrrolidone (PVPP), hydroxypropyl starch, cross-linked sodium carboxymethyl cellulose, or a combination thereof, for example. The volume of the dissolved-expansion type expander above may be increased after absorption of water, and thus the inner pressure of the pore-sealing capsule is increased.

The expander of gas generating type may include a base and an acid, and the base and the acid are isolated from each other, for example. Then, in a certain condition, water may enter the pore-sealing capsule to dissolve the acid and base out from the carriers 30. Therefore, the acid and base can react with each other to generate gas, such as CO₂, to increase the inner pressure of the pore-sealing capsule, and the pore-sealing material 20 is thus pushed out to leave the pores 10a.

The base above may be NaHCO₃, KHCO₃, or a combination thereof. The acid above may be citric acid, malic acid, tartaric acid, phosphoric acid, lactic acid, gluconic acid, glucuronic acid, vitamin C, acetic acid, salicylic acid, or a combination thereof. The isolation method of the acid and base may be respectively including the acid and the base in different carriers 30 in the pore-sealing capsule, as shown in FIG. 2, and the carriers 30 may further comprises a coating membrane to increase the storage stability. The carriers 30 may be controlled-release pellets, or fast-released pellets. The mechanisms usable for controlled release may comprise pH value or time, for example. The controlled-release pellets may be such as Bee-nest type pellets (Bio-Trend, Taiwan), Ice-mark type pellets (Bio-Trend, Taiwan), Germ type pellets (Bio-Trend, Taiwan), and Concentric type pellets (Bio-Trend, Taiwan), for example. The fast-released pellets may be Spongellets (Bio-Trend, Taiwan).

In some other embodiments, the pore-sealing material 20 may comprise a material which may be decomposed, dissolved, or melted by some other ways to open the pores 10a. Therefore, the active substances inside the pore-sealing capsule may be released from the pore-sealing capsule through the pores 10a.

In some embodiments, the pore-sealing material 20 may be decomposed by flora in the gastrointestinal tract. Accordingly, the pore-sealing material 20 may comprise a metabolizable material and a water-soluble polymer. The decomposing rate of the metabolizable material is related to the amount and activity of the flora. The water-soluble polymer is used to help the metabolizable material to form a film on the surface of the pore-sealing material 20.

Generally, the flora may be divided into probiotics and pathogens. In some embodiments, the probiotics may be Lactobacillus family, including geniuses of Lactobacillus genius and Streptococcus thermophiles. When the flora is belonged to Lactobacillus family, the metabolizable material may be carbohydrates such as glycosides, soluble cellulose, or a combination thereof.

In some other embodiments, the pathogens may be Nitrosomonas, Helicobacter pylori, E. Coli, Vibrio parahaemolyticus, Salmonella enterica, or Vibrio cholera. The metabolizable material may be the culture medium for cultivating the corresponding flora.

In some embodiments, the pore-sealing material 20 may comprise a material decomposed by a digestive enzyme in the gastrointestinal tract and a water-soluble polymer. The digestive enzyme may be secreted into different locations in the gastrointestinal tract by various glands. Accordingly, the pore-sealing material 20 may comprise a carbohydrate, a protein, or a lipid, for example. The water-soluble polymer is used to help the carbohydrate, a protein, or a lipid described above to form a film on the surface of the pore-sealing material 20.

The digestive enzyme that can digest carbohydrate includes sucrase, maltase, lactase, or isomaltase located in the front end to the rear end of the small intestine, for example. Accordingly, the carbohydrate above may be sucrose, maltose, lactose, dextrin, an oligosaccharide, a polysaccharide, or a combination thereof, for example.

The digestive enzyme that can digest protein includes pepsin, trypsin, collagenase, carboxyl peptidases A and B, amino acid peptidase, and dipeptidase, for example. Accordingly, the protein above may be a peptide, collagen, glycoprotein, or a combination thereof, for example.

The digestive enzyme that can digest lipid includes a triglyceride lipase, a phospholipid lipase, or a cholesterol lipase, for example. Accordingly, the lipid above may be a triglyceride, a phospholipid, cholesterol, fatty acids having 8-46 carbons, or a combination thereof.

In some embodiments, the pore-sealing material 20 may comprise a material disintegrated at a certain pH value in the gastrointestinal tract. Accordingly, the pore-sealing material 20 may be a derivative of cellulose, a copolymer of acrylic acid, a copolymer of methacrylic acid, a copolymer of maleic acid, a derivative of polyethylene, or a combination thereof, for example. These materials above may be decomposed or dissolved in a weak basic to basic environment, and the pores 10a are thus opened. The copolymer of methacrylic acid above may be poly(methacrylic acid-co-ethyl acrylate) 1:1 (Eugragit® L 30 D-55), which is an enteric polymer and can be dissolved at a pH value of at least 5.5.

In some embodiments, the pore-sealing material 20 may comprise a material melted at a certain temperature, such as 35-42° C., in the gastrointestinal tract. Accordingly, the pore-sealing material 20 may comprise a lipid, such as a fatty acid, a glyceride, or any combinations thereof. The fatty acid and the fatty acids of the glyceride may be saturated or unsaturated fatty acids, independently. In some embodiment, in addition to the lipid above, the pore-sealing material 20 may further comprises a surfactant to help the lipid to be disintegrated in water by emulsifying. Therefore, the leaving of the pore-sealing material 20 may be accelerated. The surfactant above may be sodium dodecyl sulfate, sodium laurate, a bile acid, a bile salt, lecithin, or a combination thereof.

In some embodiments, some of the carriers 30 in the pore-sealing capsule may comprise a mineral to increase the specific gravity of the pore-sealing capsule to be more than the specific gravity of the solution in the gastrointestinal tract. Therefore, the pore-sealing capsule may smoothly sink in the stomach, pass through the pylorus, and then reach the bottom of the intestinal track. Thereafter, the pore-sealing capsule is pushed to the rear end of the intestinal track by intestinal peristalsis. Since the mineral can let the pore-sealing capsule close to the bottom villus of the intestinal track, and thus the active substances released by the pore-sealing capsule may have a higher concentration near the bottom villus of the intestinal track to increase the absorption rate of the active substances. The mineral above may be a sodium salt, a calcium salt, a chromium salt, a zinc salt, an iron salt, a magnesium salt, a selenium salt , an aluminum salt, a silicon compound, or a combination there of.

In some embodiments, the mineral above may be ferromagnetic, paramagnetic, or diamagnetic. Therefore, the forward movement of the pore-sealing capsule may be accelerated or decelerated by an external magnetic device to keep the pore-sealing capsule staying on a certain position in the intestinal track to facilitate the controlled release of the pore-sealing capsule. The paramagnetic material may include an iron salt, and a nickel salt, for example.

In some embodiments, some carriers 30 in the pore-sealing capsule may comprise an absorbefacient to increase the absorption rate of the active substances in the pore-sealing capsule. The absorbefacient above comprises a surfactant, a fatty acid, an alcohol, an azone, chitosan, a phospholipid, a rubefacient, and piperine. The surfactant above, including an anionic surfactant (such as a bile salt) and cationic surfactant (such as a bile acid), may increase the penetration of water-soluble or lipid-soluble active substances through mucosa. The fatty acid, having 8-20 carbons, may affect the intercellular gap to increase the absorption rate of the active substances. The alcohol, the azone, and the chitosan may decrease the arrangement order of the lipid molecules in the cell membrane to facilitate the penetration of the active substances. The phospholipid, including lecithin, may increase the mucosal penetration rate of the active substances, having low water solubility but high permeability, classified in biopharmaceutical classification system II. The rubefacient, comprising ginger or oil extracts thereof, paprika or oil extracts thereof, cinnamon, peppermint oil, wintergreen oil and cinnamon oil, may increase the blood flow in the mucosa and thus increase the absorption rate of the active substances.

In light of foregoing, the material of the pore-sealing material may be chosen according to the needs and the gastrointestinal condition. Then, various action mechanisms may be used to let the pore-sealing material leave the pores of the pore-sealing capsule to reach the purpose of controlled release. Therefore, one or more suitable pore-sealing materials may be chosen and in connection with suitable size and number of the pores of the pore-sealing capsule to release one or more active substances in the pore-sealing capsule under control.

In addition, the pore-sealing capsule may contain multi-unit carriers inside the pore-sealing capsule to release multi-unit drugs of various molecular weights, pharmaceutical activities and dosages, as well as various excipients. Accordingly, after building a database of individual carriers and interaction between carriers of various drugs, foods, and diluents, a computer may be used to monitor the various properties (such as dissolution test, stability test, production feasibility, interaction between active substances and excipients, interaction between different active substances, pKa of active substances, of the pore-sealing capsule containing multi-unit carriers to obtain preliminary data. Therefore, the various costs of developing new products in the early stage may be greatly reduced, and the development speed of new products is increased.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A pore-sealing capsule released in a gastrointestinal tract under control, comprising:
a capsule shell having one or more pores;
a pore-sealing material located in at least one of the pores for sealing the pore, wherein the pore-sealing material will leave the pore when the pore-sealing capsule is located in a destination located in the gastrointestinal tract.

2. The pore-sealing capsule of claim 1, wherein the pore-sealing material comprises a material decomposed by flora in the gastrointestinal tract.

3. The pore-sealing capsule of claim 1, wherein the pore-sealing material comprises a material decomposed by a digestive enzyme in the gastrointestinal tract.

4. The pore-sealing capsule of claim 3, wherein the pore-sealing material comprises a carbohydrate, a protein, or a lipid.

5. The pore-sealing capsule of claim 1, wherein the pore-sealing material comprises a material disintegrated at a certain pH value in the gastrointestinal tract.

6. The pore-sealing capsule of claim 1, wherein the pore-sealing material comprises a material melted at a certain temperature in the gastrointestinal tract.

7. The pore-sealing capsule of claim 6, wherein the pore-sealing material comprises a lipid, a glyceride, or any combinations thereof.

8. The pore-sealing capsule of claim 7, wherein the pore-sealing material further comprises a surfactant selected from a group consisting of sodium dodecyl sulfate, sodium laurate, vitamin E, a bile acid, a bile salt, lecithin, and a combination thereof.

9. The pore-sealing capsule of claim 1, further comprising carriers located inside the capsule shell.

10. The pore-sealing capsule of claim 9, wherein at least one of the carriers comprises an expander.

11. The pore-sealing capsule of claim 10, wherein the expander is hydroxypropyl cellulose (HPC), sodium starch glycolate (SSG), polyvinylpyrrolidone (PVPP), hydroxypropyl starch, cross-linked sodium carboxymethyl cellulose, or a combination thereof.

12. The pore-sealing capsule of claim 10, wherein the expander is a base and an acid isolated from each other, the base is NaHCO₃, KHCO₃ or a combination thereof, as well as the acid is citric acid, malic acid, tartaric acid, phosphoric acid, lactic acid, gluconic acid, glucuronic acid, vitamin C, acetic acid, salicylic acid, or a combination thereof.

13. The pore-sealing capsule of claim 9, wherein at least one of the carriers comprises an absorbefacient.

14. The pore-sealing capsule of claim 13, wherein the absorbefacient is a surfactant, a fatty acid, an alcohol, an azone, chitosan, a phospholipid, a rubefacient, and piperine.

15. The pore-sealing capsule of claim 9, wherein at least one of the carriers comprises a mineral.

16. The pore-sealing capsule of claim 15, wherein the mineral is paramagnetic or diamagnetic.
